(19) United States
(12) Patent Application Publication
(10) Pub. No.: US 2007/0129617 A1
(43) Pub. Date: Jun. 7, 2007

(54) LIGHT SOURCE DRIVE ALGORITHM

(76) Inventors: Michel Noel, Sherbrooke (CA); Sylvain Dumont, St-Elie D'Orford (CA)

Correspondence Address: GOUDREAU GAGE DUBUC, 2000 MCGILL COLLEGE, SUITE 2200, MONTREAL, QC, H3A 3H3, CA

(21) Appl. No.: 11/566,291

(22) Filed: Dec. 4, 2006

Related U.S. Application Data
(60) Provisional application No. 60/741,547, filed on Dec. 2, 2005.

(51) Int. Cl. A61B 5/00

(52) U.S. Cl. 600/323

(57) ABSTRACT

The present invention relates to a method for illuminating a first and at least a second light source in a light-emitting apparatus. This method comprises the acts of illuminating the first light source for a first period during which the first light source emits a plurality of light pulses, illuminating the second light source for a second period during which the second light source emits at least one light pulse, and repeating the illuminating acts. Furthermore, the present invention relates to a light-emitting apparatus for emitting light pulses and detecting these light pulses after they have been reflected or transmitted by a substance. This apparatus comprises a first light source for illuminating the substance with a plurality of light pulses for a first period, at least a second light source for illuminating the substance with at least one light pulse for a second period, at least one photodetector for detecting the light pulses after they have been reflected or transmitted by the substance, and a means for converting the detected light pulses into at least one reading.

### CROSS REFERENCE TO RELATED APPLICATIONS

The present application claims priority from U.S. Provisional Application No. 60/741,547 filed on Dec. 2, 2005 the entirety of which is incorporated herein by reference

### FIELD OF THE INVENTION

The present invention relates generally to light source drive algorithm. In particular, the present invention relates to light source drive algorithm for use in spectrometers, photometers, pulse oxymeters, capnometers, and others similar light-emitting apparatuses.

### BACKGROUND OF THE INVENTION

The current trend in the spectrometry, photometry, pulse oxymetry, capnography and other similar light-emission techniques is to minimize power consumption without compromising the performance of the device. This trend is particularly intense in the field of pulse oxymetry.

Pulse oxymetry is a widely accepted noninvasive procedure for measuring the oxygen saturation level of a person's arterial blood, which is an indicator of their oxygen supply. Oxygen saturation monitoring is crucial in critical care and surgical applications, where an insufficient blood supply can quickly lead to injury or death. Pulse oxymetry represents at present the standard of care for the continuous monitoring of arterial oxygen saturation (SpO₂). Pulse oxymeters provide instantaneous in-vivo measurements of arterial oxygenation, and thereby can provide early warning of arterial hypoxemia, for example.

Pulse oxymeters determine the oxygen saturation level of a patient's blood, or related analyte values, based on the transmission/absorption characteristics of light transmitted through a patient's tissue. Alternatively, the oxygen saturation level of a patient's blood, or related analyte values, can be determined based on the transmission/absorption characteristics of light reflected by a patient's tissue. Similarly, spectrometers, photometers, capnometers and the like are instruments measuring the light emitted by a light source after it has been reflected or transmitted by a substance.

In general, pulse oxymeters include a probe for attaching to a patient's appendage, such as the finger, ear lobe, or nasal septum. The probe is used to emit, transmit and detect pulsed optical signals passing through the patient's tissues. In order to measure the blood oxygen level in a living body, a pulse oxymeter must distinguish two species of hemoglobin (oxyhemoglobin and deoxyhemoglobin). This is usually done by measuring the light absorption of blood at two different wavelengths at which the two hemoglobin species have substantially different absorption values. These wavelengths are typically around 605-660 nm (red visible light) and around 805-940 (infrared).

Increasingly, oxymeters are being utilized in portable, battery-operated applications. For example, a pulse oxymeter may be attached to a patient during emergency transport and remain with the patient as he is moved between hospital wards. Moreover, pulse oxymeters are often implemented as plug-in modules for multiparameter patient monitors having a restricted power budget. These applications and others create an increasing demand for lower power and higher performance pulse oxymeters.

The prior art reveals a number of more or less successful attempts to solve the power consumption problem in the field of pulse oxymetry. For example, the prior art reveals an oxymeter comprising a LED drive circuit that delivers a drive current to the LED that peaks only after the detection circuit for detecting the light has settled. Alternatively, the oxymeter can comprise a switch providing LED pulses shorter than the standard 200 ms pulses. The prior art also reveals an oxymeter having a reduced duty cycle of the LED driving circuit, such that a given LED is powered for a portion of a sampling cycle smaller than the standard 25%. It is also possible that a pulse oxymeter adjusts the driving pulse widths, frequency and amplitude to reduce power consumption. Another possibility of the prior art is to oversample the signals from the light sources to improve measurement consistency. Unfortunately, these various oxymeters have several drawbacks such as decreased measurement accuracy, decreased signal-to-noise ratio and complicated feedback circuitry.

### SUMMARY OF THE INVENTION

In order to address the above-mentioned and other drawbacks, the present invention provides a light-emitting apparatus for measuring light after it has been reflected or transmitted by a substance, and a pulse oxymeter for measuring the blood oxygen level in a human or animal patient as well as a method for operating these light-emitting apparatuses and pulse oximeters.

An object of the present invention is to provide a light-emitting apparatus for emitting light pulses and detecting these light pulses after they have been reflected or transmitted by a substance. This apparatus comprises two or more discrete lights sources. In this apparatus, the first light source illuminates the substance with a plurality of light pulses for a first period and then, the second light source illuminates the substance with at least one light pulse, or preferably a plurality of, light pulses, for a second period. These light emitting periods are consecutive to each other and are alternately repeated. The light-emitting apparatus further comprises at least one photodetector for detecting the light pulses after they have been reflected or transmitted by the substance and a means for converting the detected light pulses into at least one, or preferably a plurality of, readings.

A further object of this invention is to provide a method for illuminating a first and at least a second discrete light source in a light-emitting apparatus. The method of the present invention comprises the consecutive and alternately repeated acts of illuminating the first light source for a first period during which the first light source emits a plurality of light pulses and illuminating the second light source for a second period during which the second light source emits at least one, or preferably a plurality of, light pulses. In this method, the first and the second periods are non-overlapping.

Another object of this invention is to provide a method for detecting and converting into readings light pulses reflected or transmitted by a substance. The method of the present invention comprises the act of providing a first and at least a second discrete light source. This method further comprises the consecutive and alternately repeated acts of illuminating the substance with the first light source for a first period during which the first light source emits a plurality of light pulses, wherein the light pulses are at least partially reflected or transmitted by the substance, and illuminating the substance with the second light source for a second period during which the second light source emits at least one, or preferably a plurality of, light pulses, wherein the light pulses ar at least partially reflected or transmitted by the substance. In the method of this invention, the first and the second periods are non-overlapping. This method further comprises the acts of detecting the reflected or transmitted light pulses with at least one photodetector and converting the detected light pulse into at least one, or preferably a plurality of, readings.

Another object of this invention is to provide a pulse oximeter for emitting light pulses and detecting these light pulses after they have been reflected or transmitted by a patient's tissues. The pulse oximeter of the present invention comprises two or more lights sources emitting light at two or more different wavelengths. Preferably, these light sources are a LED emitting infrared light and another LED emitting red visible light. In the oxymeter of the present invention, the first light source illuminate the patient's tissues with a plurality of light pulses for a first period and then, the second light source illuminate the patient's tissues with at least one light pulse, or preferably a plurality of, light pulses, for a second period. These light emitting periods are consecutive to each other and are alternately repeated. The oximeter further comprises at least one photodetector for detecting the light pulses after they have been reflected or transmitted by the patient's tissues and a means for converting the detected light pulses into at least one, or preferably a plurality of, readings.

A further object of this invention is to provide a method for illuminating a first and at least a second light source in a pulse oxymeter. In this oxymeter, the first light source emits light at a wavelength different from that emitted by the second light source. The method of the present invention comprises the consecutive and alternately repeated acts of illuminating the first light source for a first period during which the first light source emits a plurality of light pulses and illuminating the second light source for a second period during which the second light source emits at least one, or preferably a plurality of, light pulses. In this method, the first and the second periods are non-overlapping.

Another object of this invention is to provide a method for detecting and converting into readings light pulses reflected or transmitted by a patient's tissues. The method of the present invention comprises the act of providing a first and at least a second light source such that the first light source emits light at a wavelength different from that emitted by the second light source. This method further comprises the consecutive and alternately repeated acts of illuminating the patient's tissues with the first light source for a first period during which the first light source emits a plurality of light pulses and illuminating the patient's tissues with the second light source for a second period during which the second light source emits at least one, or preferably a plurality of, light pulses. In the method of this invention, the first and the second periods are non-overlapping. This method further comprises the acts of detecting the reflected or transmitted light pulses with at least one photodetector and converting the detected light pulses into at least one, or preferably a plurality of, readings.

The light sources used in the present invention are conventional and are preferably light emitting diodes (LEDs), but may be any other light sources commonly used in spectrometers, photometers, pulse oxymeters, capnometers, and others similar light-emitting apparatuses.

The one or more photodetectors used in the present invention are conventional and may include any light sensitive sensor and detection circuitry commonly used in spectrometers, photometers, pulse oxymeters, capnometers, and others similar light-emitting apparatuses.

Other objects, advantages and features of the present invention will become more apparent upon reading of the following non-restrictive description of specific embodiments thereof, given by way of example only with reference to the accompanying drawings.

### BRIEF DESCRIPTION OF THE DRAWINGS

In the appended drawings:

FIG. 1 is a schematic diagram of the pulse oxymeter of the present invention;

FIG. 2 is a typical light source drive algorithm of the prior art;

FIG. 3 is a generic example of the light source drive algorithm of the present invention; and

FIG. 4 is a specific example of the light source drive algorithm of the present invention.

### DESCRIPTION OF THE ILLUSTRATIVE EMBODIMENTS

The present invention is illustrated in further details by the following non-limiting examples.

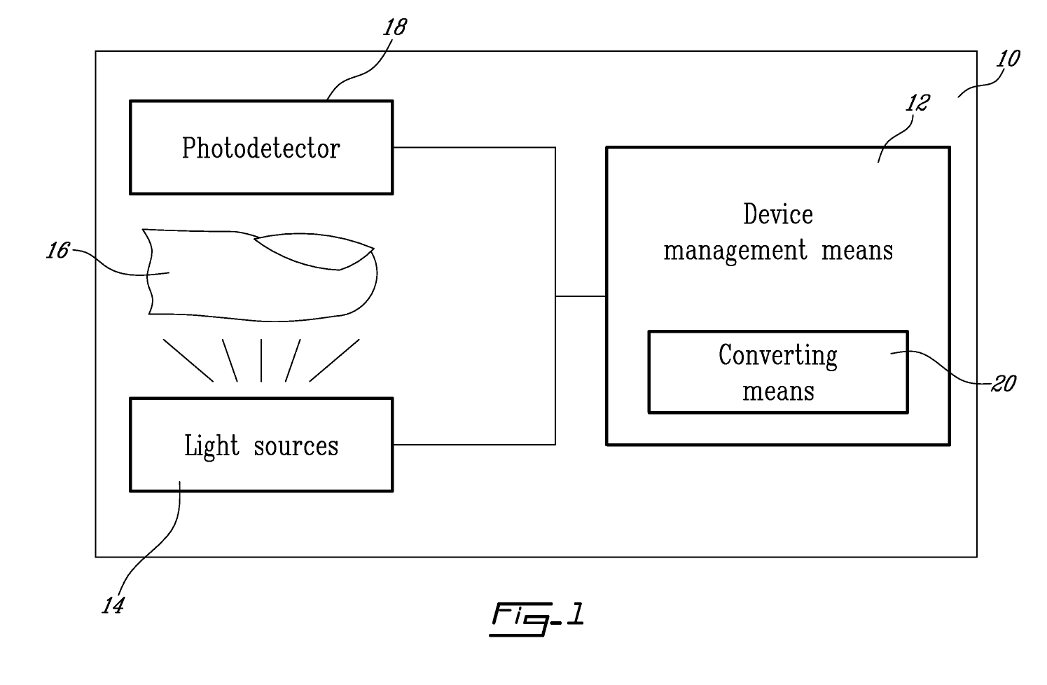

FIG. 1 shows a schematic diagram of a particular embodiment of the pulse oxymeter of the present invention, which is generally referred to using the reference numeral 10. This oxymeter is managed by device management means 12. This oxymeter comprises at least two light sources 14, one emitting infrared light and another one emitting red visible light. Theses light sources emit pulses of light of each wavelength according to the light source drive algorithm of the present invention. The light pulses emitted by the sources are, in this particular case, transmitted through a patient's tissues 16 and are then received by at least one photodetector as in 18. This photodetector measures the amount of light received and provides an output signal representative of the received optical signals. This output signal is converted into readings by converting means 20.

Example of a Typical Light Source Drive Algorithm for a Pulse Oxymeter

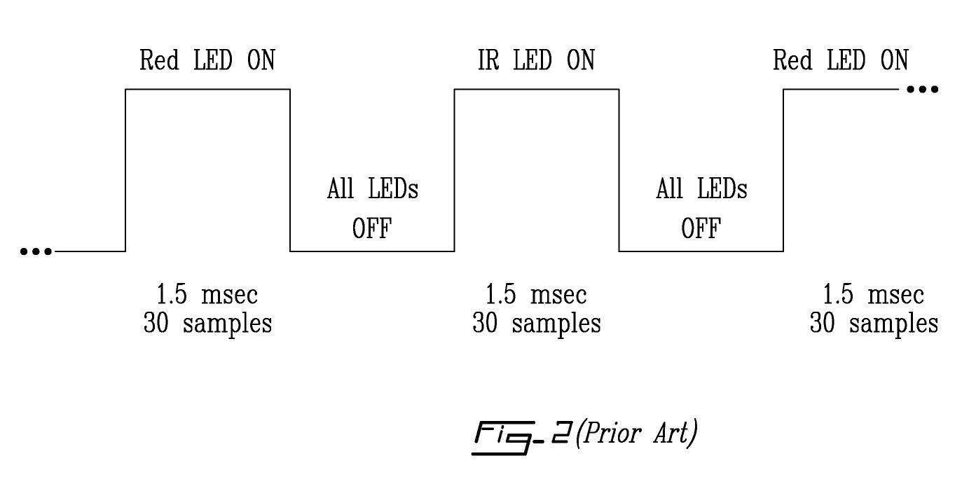

FIG. 2 shows an example of a typical light source drive algorithm of the prior art. This algorithm is given as an illustration only since the various timings indicated are different for every model of every manufacturer. In this particular case, the drive algorithm is for use in a pulse oxymeter comprising an infrared and a red LED. It is a repetitive 6 msec cycle divided into four phases:
a) red LED ON for 1.5 msec;
b) all LEDs OFF during 1.5 msec;
c) infrared LED ON during 1.5 msec; and
d) all LEDs OFF during 1.5 msec.

During this algorithm, readings can be, for example, recorded at a rate of 20 kHz. This means that up to 30 readings can be made during each of the four phases. In this configuration, the LED drive can typically require more than about 70% of the overall power consumption of the oxymeter.

Generic Example of the Light Source Drive Algorithm of the Present Invention

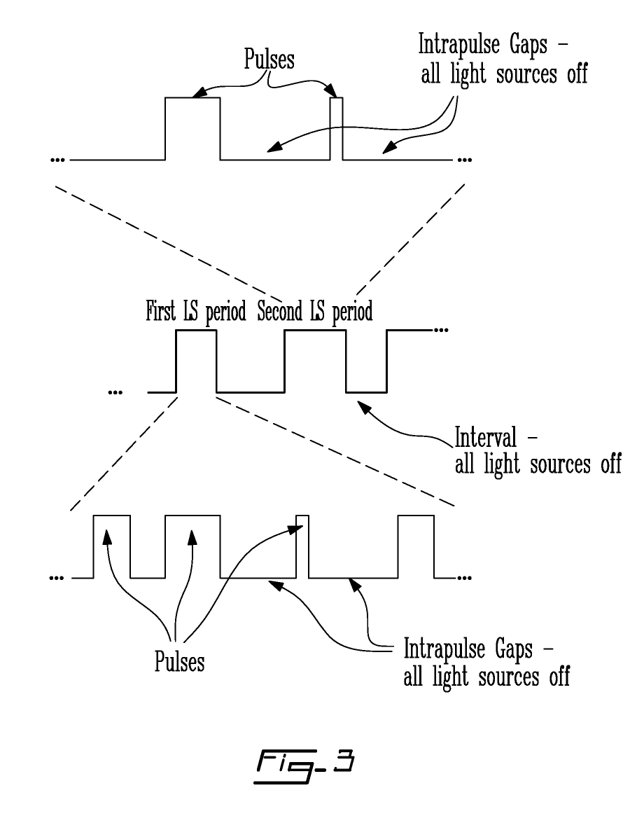

FIG. 3 shows an example of the light source drive algorithm of the present invention, which is used in the light-emitting apparatus of the present invention.

The repetitive cycle of FIG. 3 can be of any duration. It is divided into several periods, each corresponding to each of the discrete light sources (LS) of the apparatus. These periods may differ in length from one light source to the other in a given cycle and from one cycle to the other for a given period. They can be separated or not by intervals during which all light sources are off. These intervals may differ in length from one interval to the other in a given cycle and from one cycle to the other for a given interval.

Each of the periods, corresponding to each light source, comprises at least one light pulse, provided that at least one period comprises more than one pulses. These light pulses may vary in number, position and length from one pulse to the other for a given period, from one period to the other for a given cycle and from one cycle to the other for a given pulse.

The light pulses are separated by intrapulse gaps where all light sources are off. These intrapulse gaps may vary in length from one gap to the other for a given period, from one period to the other for a given cycle and from one cycle to the other for a given gap. As a result, the light pulses can be equally or non-equally spaced independently during any period.

During each pulse, interval and intrapulse gap, a number of readings can be recorded. These readings may vary in length, number, spacing, etc. from one pulse/intrapulse gap to the other in a given period, from one period to the other in a given cycle, from one interval to the other in a given cycle, and from one cycle to the other for a given pulse, interval or intrapulse gap. For example, the readings can be equally spaced, non-equally spaced or recorded as fast as possible anywhere during the light pulse, interval and/or intrapulse gap, they may be spaced using a predetermined timing pattern, or they may be recorded only after a certain waiting time after the beginning of a light pulse, interval and/or intrapulse gap.

Specific Example of the Light Source Drive Algorithm of the Present Invention

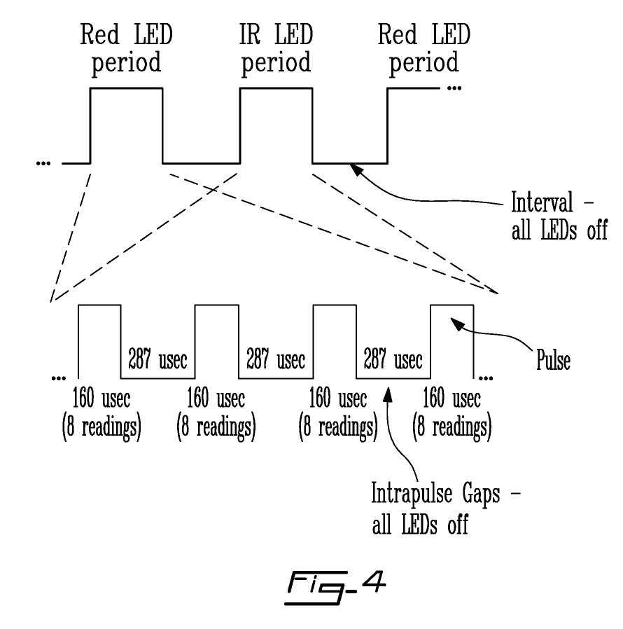

FIG. 4 shows a particular embodiment of the light source drive algorithm of the present invention. In the particular case, the algorithm is used for illuminating two LEDs in a pulse oxymeter of the present invention. This particular oxymeter comprises two LEDs emitting infrared and red light, respectively. This algorithm is a repetitive 6 msec cycle comprising one period for each of the LED of the oxymeter.

In this particular case, each period lasts 1.5 msec during which the infrared or the red LED emits a plurality of light pulses. These light pulses lasts 160 usec each and are separated by intrapulse gaps, lasting 287 usec, where all LED are off. The periods are separated by intervals lasting 1.5 msec during which all LEDs are off.

In this particular case, readings are recorded at the rate of 50 kHz during each 160 usec light pulse, and thus, 8 equally spaced readings are recorded during each pulse.

When the pulse oxymeter of FIG. 1 is operated using the light source drive algorithm of FIG. 4, the energy consumption of the oximeter is reduced comparatively to that of the same oxymeter operated using the algorithm of FIG. 2.

Although the present invention has been described hereinabove by way of specific embodiments thereof, it can be modified, without departing from the spirit and nature of the subject invention as defined in the appended claims.

What is claimed is:

1. A method for illuminating a first and at least a second light source in a light-emitting apparatus, the method comprising the acts of:
illuminating the first light source for a first period during which the first light source emits a plurality of light pulses;
illuminating the second light source for a second period during which the second light source emits at least one light pulse; and
repeating said illuminating acts;
wherein said first and second periods are non-overlapping.

2. The method as claimed in claim 1, wherein, during said second light source illuminating act, the second light source emits a plurality of light pulses.

3. The method as claimed in claim 1, further comprising at least one interval between the illuminating acts during which none of the light sources is illuminated.

4. The method as claimed in claim 1, further comprising, prior to said repeating act, the act of illuminating at least one additional light source for at least one additional period during which said additional light source emits at least one light pulse, wherein each of said at least one additional periods and said first and second periods are non-overlapping.

5. A method for detecting and converting into readings light pulses reflected or transmitted by a substance, the method comprising the acts of:
providing a first and at least a second light sources;
illuminating the substance with said first light source for a first period during which the first light source emits a plurality of light pulses, wherein said light pulses are at least partially reflected or transmitted by the substance;
illuminating the substance said second light source for a second period during which the second light source emits at least one light pulse wherein said light pulses are at least partially reflected or transmitted by the substance;
repeating said illuminating acts,
detecting said reflected or transmitted light pulses with at least one photodetector; and
converting said detected light pulses into a least one reading;
wherein said first and second periods are non-overlapping and wherein said detecting and converting acts are performed concurrently to said illuminating and repeating acts.

6. The method as claimed in claim 5, wherein, during said converting act, a plurality of readings is provided for each light pulse.

7. The method as claimed in claim 5, further comprising the act of:
detecting light with at least one photodetector during an interval, between the illuminating acts, during which none of the light sources is illuminated; and
converting said detected light into a least one reading.

8. The method as claimed in claim 5, further comprising the act of:
detecting light with at least one photodetector during at least one intrapulse gap, between the light pulses, during which none of the light sources is illuminated; and converting said detected light into a least one reading.

9. A light-emitting apparatus for emitting light pulses and detecting said light pulses after they have been reflected or transmitted by a substance, the light-emitting apparatus comprising:
a first light source for illuminating the substance with a plurality of light pulses for a first period;
at least a second light source for illuminating the substance with at least one light pulse for a second period;
at least one photodetector for detecting said light pulses after they have been reflected or transmitted by the substance; and
a means for converting said detected light pulses into at least one reading;
wherein all the periods during which said light pulses are emitted are consecutive to each other and alternately repeated.

10. The light-emitting apparatus as claimed in claim 9, wherein there is, between the periods during which said light pulses are emitted, at least one interval during which no light is emitted by the light sources.

11. The light-emitting apparatus as claimed in claim 9, wherein one of the light sources is a LED emitting infrared light and the other light source is a LED emitting red visible light.

12. The light-emitting apparatus as claimed in claim 9, wherein the second light source emits a plurality of light pulses.

13. The light-emitting apparatus as claimed in claim 9, comprising more than two light sources.

14. The light-emitting apparatus as claimed in claim 9, comprising more than one photodetector.

15. The light-emitting apparatus as claimed in claim 9, wherein a plurality of readings is provided for each light pulse.

16. The light-emitting apparatus as claimed in claim 9, wherein the photodetector detects light during at least one interval, between the periods during which said light pulses are emitted, during which no light is emitted by the light sources and wherein at least one reading is provided for at said at least one interval.

17. The light-emitting apparatus as claimed in claim 9, wherein the photodetector detects light during at least one interval intrapulse gap, between the light pulses, during which no light is emitted by the light sources and wherein at least one reading is provided for said at least one intrapulse gap.

18. A method for illuminating a first and at least a second light source in a pulse oxymeter, the first light source emitting light at a wavelength different from that emitted by the second light source, the method comprising the acts of:
illuminating the first light source for a first period during which the first light source emits a plurality of light pulses;
illuminating the second light source for a second period during which the second light source emits at least one light pulse; and
repeating said illuminating acts;
wherein said first and second periods are non-overlapping.

19. The method as claimed in claim 18, wherein, during said second light source illuminating act, the second light source emits a plurality of light pulses.

20. The method as claimed in claim 18, further comprising at least one interval between the illuminating acts during which none of the light sources is illuminated.

21. The method as claimed in claim 18, further comprising, prior to said repeating act, the act of illuminating at least one additional light source for at least one additional period during which said additional light source emits at least one light pulse, wherein each of said at least one additional periods and said first and second periods are non-overlapping.

22. A method for detecting and converting into readings light pulses reflected or transmitted by a patient's tissues, the method comprising the acts of:
providing a pulse oxymeter comprising a first and at least a second light sources;
illuminating the patient's tissues with said first light source for a first period during which the first light source emits a plurality of light pulses, wherein said light pulses are at least partially reflected or transmitted by the patient's tissues;
illuminating the patient's tissues with said second light source for a second period during which the second light source emits at least one light pulse, wherein said light pulses are at least partially reflected or transmitted by the patient's tissues;
repeating said illuminating acts,
detecting said reflected or transmitted light pulses with at least one photodetector; and
converting said detected light pulses into a least one reading,
wherein said first and second periods are non-overlapping and wherein said detecting and converting acts are performed concurrently to said illuminating and repeating acts.

23. The method as claimed in claim 22, wherein, during said converting act, a plurality of readings is provided for each light pulse.

24. The method as claimed in claim 22, further comprising the act of:
detecting light with at least one photodetector during an interval, between the illuminating acts, during which none of the light sources is illuminated; and
converting said detected light into a least one reading.

25. The method as claimed in claim 22, further comprising the act of:
detecting light with at least one photodetector during at least one intrapulse gap, between the light pulses, during which none of the light sources is illuminated; and
converting said detected light into a least one reading.

26. A pulse oximeter for emitting light pulses and detecting said light pulses after they have been reflected or transmitted by a patient's tissues, the pulse oxymeter comprising:
a first light source for illuminating the patient's tissues with a plurality of light pulses for a first period;
at least a second light source for illuminating the patient's tissues with at least one light pulse for a second period;
at least one photodetector for detecting said light pulses after they have been reflected or transmitted by the patient's tissues; and
a means for converting said detected light into at least one reading;
wherein said first light source emits light at a wavelength different from that emitted by said second light source and wherein all the periods during which said light pulses are emitted are consecutive to each other and alternately repeated.

27. The pulse oximeter as claimed in claim 26, wherein there is, between the periods during which said light pulses are emitted, at least one interval during which no light is emitted by the light sources.

28. The pulse oximeter as claimed in claim 26, wherein one of the light sources is a LED emitting infrared light and the other light source is a LED emitting red visible light.

29. The pulse oximeter as claimed in claim 26 wherein the second light source emits a plurality of light pulses.

30. The pulse oximeter as claimed in claim 26, comprising more than two light sources.

31. The pulse oximeter as claimed in claim 26, comprising more than one photodetector.

32. The pulse oximeter as claimed in claim 26, wherein a plurality of readings is provided for each light pulse.

33. The pulse oximeter as claimed in claim 26, wherein the photodetector detects light during at least one interval, between the periods during which said light pulses are emitted, during which no light is emitted by the light sources and wherein at least one reading is provided for said at least one interval.

34. The pulse oximeter as claimed in claim 26, wherein the photodetector detects light during at least one interval intrapulse gap, between the light pulses, during which no light is emitted by the light sources and wherein at least one reading is provided for said at least one intrapulse gap.
